(19) United States
(12) Patent Application Publication
(10) Pub. No.: US 2013/0116645 A1
(43) Pub. Date: May 9, 2013

(54) Absorbent Foam Tape And Related Methods Thereof

(71) Applicant: Kevin M. Corley, Reading, MA (US)

(72) Inventor: Kevin M. Corley, Reading, MA (US)

(73) Assignee: Tyco Healthcare Group LP, Mansfield, MA (US)

(21) Appl. No.: 13/290,283

(22) Filed: Nov. 7, 2011

(51) Int. Cl. A61F 13/02; A61L 15/22; B32B 38/00; B32B 37/02; B32B 37/14

(52) U.S. Cl. 604/369; 156/60; 156/185; 156/281

(57) ABSTRACT

A foam tape having an adhesive layer for positioning against a tissue surface, a porous layer including a plurality of pores adapted to absorb fluid, a breathable layer configured to allow the passage of vapor therethrough, and an optional release layer is disclosed. The foam tape may be utilized as a wound dressing or in a compression bandage system.

### BACKGROUND

1. Field

The present disclosure relates generally to the treatment of wounds, and in particular to rolled absorbent foam tapes suitable for use as a wound dressing, as a bandage, or as a layer in a compression dressing or negative wound pressure therapy system.

2. Related Art

Wound dressings have been used in the medical industry to protect and/or facilitate healing of open wounds. Wound dressings may include an absorbent pad to provide both cushioning to the wound site and assist in the collection of exudates from the wound. Wound dressings may also be utilized in compression bandage systems to treat medical conditions such as venous stasis ulcers and lymphedema.

In the case of deep wounds, one technique has been to use negative wound pressure therapy (NWPT), which is also known as suction or vacuum therapy. Application of negative pressure, reduced, or sub-atmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound, such as exudates, which may tend to harbor bacteria. A variety of negative pressure devices have been developed to allow excess wound fluids, such as exudates, to be removed while at the same time isolating the wound to protect the wound and, consequently, affect recovery time. Generally, these devices provide for a wound to be covered to facilitate suction at the wound area. For example, Vess discloses, in U.S. Pat. No. 7,825,289 B2, a wound dressing adhesive compression device.

### SUMMARY

In accordance with one or more aspects, the present invention can provide, or facilitate wound treatment with a versatile wound dressing which may be utilized to treat open wounds, to cushion tissue requiring compression, and/or to supplement the adhesion of a dressing system.

Some aspects of the invention can be directed to a foam tape that can be adapted and configured to provide improved absorbency, softness, conformability, and antimicrobial efficacy to facilitate the treatment of wounds.

In still other aspects of the invention, the foam tape may be used for a variety of surgical and wound applications including, for example, to close and heal tissue defects, incisions, and wounds, as well as for cushioning tissue and/or supplementing the adhesion of a wound dressing system.

One or more aspects of the invention can be directed to a method of preparing a wound dressing. The method can comprise providing a tape comprising a foam layer having a first surface and an opposite surface, an adhesive layer disposed in a predetermined adhesive pattern on at least a portion of the first surface of the foam layer, a breathable layer disposed on at least a portion of the opposite surface of the foam layer, and a release layer disposed on at least one of the breathable layer and the adhesive layer. In some embodiments of the invention, the method can further comprise wrapping at least a portion of the tape around a core or a spindle to produce a foam roll. The foam layer can be comprised of polyurethane with open cells. The release layer can be comprised of a material selected from the group consisting of a wax, a polyolefin, a silicone, a vinyl polymer, and combinations thereof. In some embodiments of the invention, providing the tape can comprise introducing at least one therapeutic agent in the foam layer. In some further embodiments of the invention, providing the tape can comprise disposing the adhesive layer on the at least a portion of the first surface of the foam layer in a discontinuous adhesive pattern. In still further embodiments of the invention, providing the tape can comprise disposing a material selected from the group consisting of a silicone, an acrylic, and a natural rubber on the at least a portion of the first surface of the foam layer to produce the predetermined adhesive pattern. The method, in accordance in one or more embodiments of the invention, can further comprise sterilizing the tape. In yet further embodiments of the invention, the release layer can comprise a material selected from the group consisting of a wax, a polyolefin, a silicone, a vinyl polymer, and combinations thereof.

One or more aspects of the invention can be directed to a foam tape, which can be wrapped around a core. In one or more embodiments pertinent to one or more such aspects of the invention, the foam tape can comprise at least one absorbent foam layer including a plurality of pores. The absorbent foam layer typically has a first surface and a second surface. The foam tape can further comprise at least one adhesive layer that can be disposed in a predetermined pattern on at least a portion of the first surface of the absorbent foam layer. In some embodiments of the invention, the foam tape can comprise a breathable layer disposed on at least a portion of the second surface of the absorbent foam layer. In further embodiments of invention, the foam tape can further comprise a release layer disposed on at least a portion of the breathable layer. The absorbent foam layer can be comprised of polyurethane. The release layer can be comprised of a material selected from the group consisting of a wax, a polyolefin, a silicone, a vinyl polymer, and combinations thereof. In further embodiments of the invention, the foam tape can comprise at least one therapeutic agent, typically in the absorbent foam layer. One or more embodiments of the invention can involve having the adhesive layer that is disposed on the at least a portion of the first surface of the absorbent foam layer in a discontinuous predetermined pattern. The adhesive layer, in some embodiments of the invention, can be comprised of a material selected from the group consisting of a silicone and an acrylic. In some variants of some embodiments of the invention, the foam tape can have a thickness in a range of from about 0.1 inch to about 0.3 inch.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1A is schematic illustration showing a perspective view of a foam tape in accordance with one or more embodiments of the present invention;

FIG. 1B is a schematic illustration showing a cross-section of the foam tape exemplarily illustrated in FIG. 1A;

FIG. 1C is a schematic illustration showing a cross-section of an alternate embodiment of the foam tape exemplarily illustrated in FIG. 1B;

FIG. 2A is a schematic illustration showing a cross-section of a compression bandage system including a foam tape layer in accordance with one or more embodiments of the present invention; and

FIG. 2B is a schematic illustration showing a perspective view of a limb wrapped in a compression bandage system exemplarily illustrated in FIG. 2A.

### DETAILED DESCRIPTION

In accordance with one or more aspects of the invention, the present disclosure can provide a wound dressing which includes from a distal surface to a proximal surface, for example, an adhesive layer for positioning against a tissue surface, a porous layer with a plurality of pores adapted to absorb at least a portion of a fluid from a wound, an optional breathable layer configured to inhibit passage of aqueous fluids and allow the passage of vapor therethrough, and a release layer having a lower coefficient of friction than the breathable layer. The optional breathable layer can be comprised of a non-porous membrane.

In accordance with one or more aspects of the invention, the present disclosure can provide a compression bandage comprising a wound dressing, which may be utilized as a base layer, and a compression layer including an elastic member. The compression bandage can comprise a tape comprising an absorbent foam layer including a plurality of pores, the absorbent foam layer having a first surface and a second surface; an adhesive layer disposed in a predetermined pattern on at least a portion of the first surface of the absorbent foam layer; and a breathable layer disposed on at least a portion of the second surface of the absorbent foam layer.

In accordance with some aspects of the invention, the present disclosure can provide a membrane layer. In some aspects of the invention, the present disclosure can be directed to a wound therapy apparatus comprising the membrane layer. The wound therapy apparatus in accordance with one or more embodiments of the disclosure typically includes the membrane layer and a vacuum source or a source of sub atmospheric pressure. The membrane layer can be dimensioned for positioning relative to a wound and can be adapted to form a substantially fluid-tight seal around the wound to define a reservoir in which a sub atmospheric pressure may be applied and preferably maintained. Typically, the vacuum source is in fluid communication with the reservoir and is configured to provide an appropriate sub atmospheric pressure to the reservoir, which can stimulate healing of the wound.

In some embodiments in accordance with some aspects of the invention, the tape can be utilized in a wound dressing as, for example, a spacer in a sub atmospheric pressure wound therapy apparatus. In one or more such embodiments of some aspects of the invention, the sub atmospheric pressure wound therapy apparatus typically includes a membrane layer, a vacuum source, the spacer, and a compression bandage. The spacer is typically disposed over an upper surface of the membrane layer and, in some cases, around the periphery of the wound. The compression bandage can be configured to apply a compressive force to the spacer such that the spacer facilitates distribution of at least a portion of the compressive force to the membrane layer which can reinforce the fluid-tight seal. An example of a sub atmospheric pressure wound therapy apparatus is disclosed in U.S. Pat. No. 7,825,289 B2, the entire content of which is incorporated herein by reference for all purposes, including, but not limited to, pertinent to embodiments utilizing any one or more of the various tapes as a compression member therefor.

In the drawings and in the description which follows, in which like references numbers identify similar or identical elements, the term “proximal” will refer to the end of an article or device that is closest to the clinician during use, while the term “distal” will refer to the end that is furthest from the clinician. As used herein, the term “patient” should be understood as referring to a human subject or other animal, and the term “clinician” should be understood as referring to a doctor, nurse, or other care provider and may include support personnel. In some cases, the term distal refers a side or a surface of an article or device that is in contact with a skin surface of a patient.

One or more aspects of the invention can be directed to a foam tape, which can be wrapped around a core. In one or more embodiments pertinent to one or more aspects of the invention, the foam tape can consist of or consist essentially of an absorbent foam layer including a plurality of pores, an adhesive layer, a breathable layer, and a release layer. In one or more embodiments pertinent to one or more further aspects of the invention, the foam tape can consist of or consist essentially of an absorbent foam layer and an adhesive layer. In one or more embodiments pertinent to one or more still further aspects of the invention, the foam tape can consist of or consist essentially of an absorbent foam layer, an adhesive layer, and a release layer. In one or more embodiments pertinent to one or more yet further aspects of the invention, the foam tape can consist of or consist essentially of an absorbent foam layer, an adhesive layer, and a breathable layer. The adhesive layer can be disposed in a predetermined pattern on at least a portion of a first surface of the absorbent foam layer. The breathable layer can be disposed on at least a portion of the second surface of the absorbent foam layer. The release layer can be disposed on at least a portion of the breathable layer. In yet further embodiments of the invention, the absorbent foam layer can consist of or consist essentially of polyurethane foam. The release layer can be a material selected from the group consisting of poly(ethylene terephthalate), polyethylenes, polycarbonates, wax, silicones, vinyl polymers, and combinations thereof.

In further embodiments of the invention, the foam tape can comprise at least one therapeutic agent in the absorbent foam layer. One or more embodiments of the invention can involve having the adhesive layer that is disposed on the at least a portion of the first surface of the absorbent foam layer in a discontinuous predetermined pattern. The adhesive layer, in some embodiments of the invention, can be comprised of a material selected from the group consisting of a silicone, an acrylic, and a natural rubber. For example, in one or more embodiments pertinent to one or more aspects of the invention, the foam tape can consist of or consist essentially of an absorbent foam layer, an adhesive layer, a breathable layer, a therapeutic agent, and a release layer. In one or more embodiments pertinent to one or more further aspects of the invention, the foam tape can consist of or consist essentially of an absorbent foam layer, an adhesive layer, a therapeutic agent, and a release layer. In one or more embodiments pertinent to one or more aspects of the invention, the foam tape can consist of or consist essentially of an absorbent foam layer, an adhesive layer, a breathable layer, and a therapeutic agent.

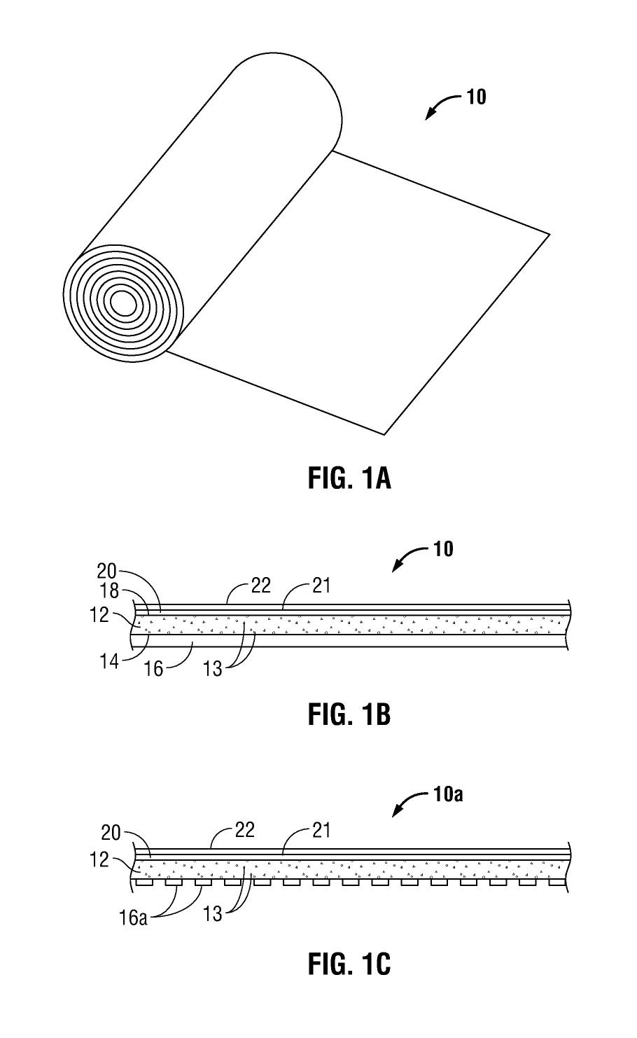

Referring to FIGS. 1A and 1B, an absorbent foam tape in accordance with some embodiments pertinent to one or more aspects of the invention is referenced as 10, which is exemplarily illustrated as a roll that is optionally wrapped around a core. Foam tape 10 typically includes at least one foam or porous layer 12 including a first, distal surface 14, at least a portion thereof can have or be coated with at least one adhesive layer 16, and a second, opposite, proximal surface 18, at least a portion of which can optionally have or be adjacent one or more breathable layers 20. In an optional variant in accordance with some embodiments of the invention, tape 10 can comprise a release layer 22, which can be disposed on at least a portion of the one or more breathable layers, or in some cases, can be disposed against the at least one adhesive layer 16.

Porous layer 12 typically has a plurality of pores 13, over at least a portion thereof, to facilitate absorption of fluids, such as physiological fluids and wound exudates. Pores 13 may be dimensioned and configured to absorb at least a portion of any fluids away from the first or distal surface. For example, at least a portion of pores 13 can effect wicking or transport of at least a portion of fluid from a wound bed into porous layer 12. Any of pores 13 can have a pore size that at least partially influences the fluid flow resistance through porous layer 12 and the fluid retention within porous layer 12 thereby facilitating control of the amount of moisture in the healing environment. In some embodiments pertinent to some aspects of the invention, porous layer 12 can have a plurality of pores in a plurality of pore sizes. The pores in any portion of layer 12 can have a normal distribution of pore sizes, a bimodal distribution of pore sizes, or a multimodal distribution of pore sizes.

In some embodiments pertinent to some aspects of the invention, porous layer 12 can have a gradient of pore size. For example, the pore size, or range of pore sizes, can increase from the outer surfaces to a central region of the porous layer. In other variants of such embodiments, the pore size or range of pore sizes can increase from the first surface to the opposite surface. The gradient of pore sizes can be linearly dependent on the depth or thickness of porous layer 12. In some cases, the gradient can be exponentially dependent on the depth or thickness of porous layer 12. Other gradient profiles are contemplated in variants of such embodiments of the invention. The pore size or range of pore sizes can increase from outer edges of tape 10 to an inner region thereof. In yet other cases, the pore size or range of pore sizes can increase from one edge to the opposite edge of tape 10. The porous layer can, in some embodiments of the invention, have a gradient of pore sizes or range of pore sizes that can vary linearly, geometrically, or exponentially relative to a distance from an edge of tape 10 to the opposite edge, to the central region, and/or to the inner region of the tape.

The density of pores in porous layer 12 can be uniform throughout the depth or thickness thereof. In other cases, porous layer 12 can have a pore density that increases from the outer surfaces to the central region of the porous layer. The pore density gradient can increase linearly. In some variants of some embodiments of the invention, the pore density can vary geometrically or exponentially relative of depth of porous layer 12. The density of pores 13 in porous layer 12 thus facilitates tailoring the stiffness of the various articles of the invention, and can thus, provide various levels of comfort of the foam tape 10. Still further embodiments of the invention contemplate having a tape 10 with increasing pore densities from outer edges to an inner region thereof. In other configurations in accordance with some aspects of the invention, the pore densities can increase from one edge to the opposite edge of tape 10. The porous layer, in some embodiments of the invention, can have a gradient of pore density that can vary linearly, geometrically, or exponentially relative to a distance from an edge of the tape to the opposite edge, to the central region, and/or to the inner region of the tape.

In some embodiments pertinent to some aspects of the invention, openings or pores 13 may be in sufficient number and size so as to interconnect across the entire thickness of porous layer 12.

Suitable materials that can be used as porous layer 12 include, but are not limited to, fibrous structures such as knitted substrates, woven substrates, non-woven substrates as well as foams such as open or closed cell foams. Woven fabrics, knitted fabrics, and open cell foam are illustrative examples of structures in which pores 13 can be in sufficient number and size so as to interconnect across the entire thickness or at least a portion of porous layer 12. In further embodiments of the invention, pores 13 do not interconnect across the entire thickness of or across any portion of porous layer 12, but can be interconnected through a portion of the thickness thereof or across a portion of the width thereof. In some embodiments pertinent to some aspects of the invention, pores 13, or a portion thereof, can be located on a portion of a surface, such as from a distal surface and/or proximal surface 14, 18, of porous layer 12. Other portions of the porous layer 12 can thus be non-porous or be pore-free. It is envisioned that pores 13 may be arranged in any manner in porous layer 12. For example, pores 13 may be configured in a random or uniform manner, or define a predetermined pore pattern.

Porous layer 12 may include agents such as medicaments or therapeutic agents, bonded or coated thereto and/or therein to reduce bioburden in the wound, promote healing, and reduce pain associated with changes or removal of foam tape 10. Medicaments or therapeutic agents that can be utilized include, for example, antimicrobial agents, growth factors, antibiotics, and analgesics. Furthermore, when an analgesic is used in any one or more embodiments of the invention, the analgesic may include a mechanism that would allow the preferred or controlled release of that agent prior to dressing removal or change. For example, release of the therapeutic agent may be initiated by exposure to a triggering environment such as an elevated moisture or humidity level of at least about 60% relative humidity. Other triggering conditions may include exposure to energy such as actinic radiation that converts, such as by cleaving, one or more precursor compounds into a therapeutic agent in its bioactive structure.

The concentration of the antimicrobial agent in the porous layer can range from about 0.1% to about 0.8%, by dry weight of the porous layer. For example, some particular embodiments of the invention can involve utilizing polyhexamethylene biguanide (PHMB) as an at least one antimicrobial agent with a concentration in the porous layer in a range of from about 0.35% (3500 ppm) to about 0.65% (6500 ppm). The normal operating range for PHMB concentration in the finished product is typically about 0.5%±0.15% based on the “dry” weight of the porous layer.

In embodiments wherein at least a portion of porous layer 12 comprises a fibrous material, the fibers may be filaments or threads suitable for knitting or weaving or may be staple fibers, such as those frequently used for preparing non-woven materials. The fibers may be made from any biocompatible material. Thus, the fibers may be formed from a natural material or a synthetic material. The material from which the fibers are formed may be bioabsorbable or non-bioabsorbable. It is understood that any combination of natural, synthetic, bioabsorbable and non-bioabsorbable materials may be used to form the fibers. Porous layer 12 may be formed using any method suitable to forming fibrous structures, including but not limited to knitting, weaving, non-woven techniques, wet-spinning, electrospinning, extrusion, co-extrusion. The term biocompatible refers to the ability of a material or component to perform its intended function with respect to a medical therapy without eliciting any undesirable local or systemic effects in the recipient or beneficiary of that therapy, generating at least one desired appropriate beneficial cellular or tissue response in that specific situation, and, in some cases, improving the clinically relevant performance of that therapy.

In embodiments wherein at least a portion of porous layer 12 comprises a foam, porous layer 12 may be formed using any method suitable to forming a foam or sponge including, but not limited to, the lyophilization or freeze-drying of a composition. The foam may be cross-linked or non-cross-linked. In some embodiments pertinent to some aspects of the invention, porous layer 12 may be an open cell polyurethane foam. A foam that can be utilized in some embodiments of the invention can be produced utilizing mixture comprising an aqueous phase, a prepolymer phase, and optionally, an therapeutic agent phase, pumped to a mixing head where they are mixed together to produce the foam. The aqueous media can comprise, for example, about 98.5% water, about 0.5% surfactant such as those commercially available under the EMULGADETM brand from Cognis Corporation, and about 1.5% surfactant, such as those commercially available under the GLUCOPON™ brand from Cognis Corporation. The prepolymer phase can comprise at least one prepolymer, monomer, or polymer precursor, such as HYPOL™ 2002 hydrophilic polyurethane prepolymer from The Dow Chemical Corporation, Midland, Mich. The therapeutic agent phase can comprise an antimicrobial agent, such as about 20% polyhexamethylene biguanide (PHMB) and 80% water, which is commercially available as COSMOCIL™ CQ, from Arch Chemicals, Inc. Norwalk, Conn.

At least a portion of distal surface 14 of porous layer 12 is typically coated with at least one adhesive layer 16 to aid in the attachment of porous layer 12 to tissue. Adhesive layer 16 may comprise a biocompatible adhesive such as those fabricated from silicone, acrylic, and rubber. In some embodiments pertinent to some aspects of the invention, adhesive layer 16 may include, but is not limited to, adhesives which cure upon tissue contact, which cure or crosslink upon exposure to ultraviolet (UV) light, which are pressure sensitive, or which are any combinations thereof.

The adhesive layer may be applied in a predetermined pattern on the at least a portion of the distal surface of the porous layer. For example, as illustrated in FIG. 1C, portions of porous layer 12 can be free of adhesive material of the least one adhesive layer 16a to facilitate fluid uptake therethrough into porous layer 12. Adhesive layer 16a may be applied to porous layer 12 in any predetermined pattern or design to cover the desired surface area of distal surface 14. For example, discontinuous regions proximate outer sides or edges of a surface of porous layer can have the at least one adhesive layer, and central regions distal from the outer sides or edges of the surface can be free of the adhesive material.

Further embodiments of the invention can involve utilizing a film adhesive layer that is comprised of, or consists of, a polyurethane film and an adhesive material on at least a portion of one or more sides of the polyurethane film. The polyurethane film can be a breathable material. In some configurations, perforated adhesive layer can comprises a first adhesive material, such as a skin friendly copolymer acrylic adhesive such as GMS 2495 pressure sensitive adhesive, available from Cytec Industries Inc., Woodland Park, N.J. Other skin friendly adhesives could be utilized in this construction. The side of the polyurethane film, typically the side that adheres to the foam can be at least partially coated with a second adhesive material, such as a copolymer acrylic adhesive, such as GMS 3101-03 pressure sensitive adhesive, also available from Cytec Industries Inc. In other cases, the second adhesive material can be high shear adhesive. The entire layer or a portion thereof can have perforations therethrough.

Proximal surface 18 can comprise a breathable layer 20. Breathable layer 20 can comprise a substantially closed surface, such as a film, that at least partially inhibit the passage of aqueous fluid therethrough, but can have desirable vapor transport characteristics to be breathable. Thus in some embodiments of the invention, layer 20 can be at least partially permeable to vapor, such as moisture from a wound, while being at least partially impermeable to air and aqueous fluids. Breathable layer 20 can serve, in some configurations pertinent to some aspects of the invention, as a barrier that at least partially prevents or at least inhibits contaminants from entering a wound and allow physiological derived fluids from exiting therethrough. Breathable layer 20 may be made from any biocompatible natural and/or synthetic material that may be the same or different from the material of porous layer 12.

Release layer 22 can be coated on a proximal surface 21 of breathable layer 20 or be disposed against the at least one adhesive layer 16. Release layer 22 typically has a lower coefficient of friction, compared to breathable layer 20, which can reduce the friction between breathable layer 20 and any materials or surface that can contact breathable layer 20. Release layer 22 can have a slick or silky characteristic thereby providing a desirable finish on foam tape 10. Release layer 22 may be a continuous or discontinuous layer of at least one release agent such as, but not limited to, hydrocarbon waxes, polyolefins, silicone and/or vinyl based polymers, copolymers, and combinations thereof. Release layer 22 can have an area profile that at least partially corresponds with an area profile of adhesive layer 16.

Foam tape 10 may be cast at a thickness of from about 2.5 mm (about 0.1 inch) to about 5 mm (about 0.2 inch). In some embodiments pertinent to some aspects of the invention, the foam tape can have a thickness of about 4 mm (about 0.16 inch). In some configurations pertinent to some aspects of the invention, foam tape 10 may have a thickness less of than about 2.5 mm (about 0.1 inch) which can be utilized as a low profile wrap. Other configurations pertinent still further aspects of the invention can involve a tape having a thickness of at least about than about 5 mm (about 0.2 inch).

As illustrated in FIG. 1A, foam tape 10 may be configured as a roll with any desirable width and length. For example, in some embodiments pertinent to some aspects of the invention, tape 10 may be assembled into rolls having a width of about 50 mm (about 2 inches). In roll form, release layer 20 can ease unwinding of tape 10.

The soft and conformable nature of tape 10 can provide comfort or at least reduce discomfort against sensitive wounds. The absorbent and adhesive nature of the tape can serve as a wound dressing and covering layer by combining the desirable features of a foam and with desirable feature of a tape. The antimicrobial efficacy of the tape can facilitate a well-balanced wound healing environment. Moreover, the various widths, lengths, and shapes of the tape 10 allows a clinician to customize dressings for use with tissue with irregular surfaces, such as fingers, feet, ankles, elbows, and sacral areas.

The tape may also be utilized to cushion a wound and/or to supplement the adhesion of an additional dressing. For example, tape 10 can be wrapped around a patient's limb in one or a plurality of layers to provide support and compressive force thereon.

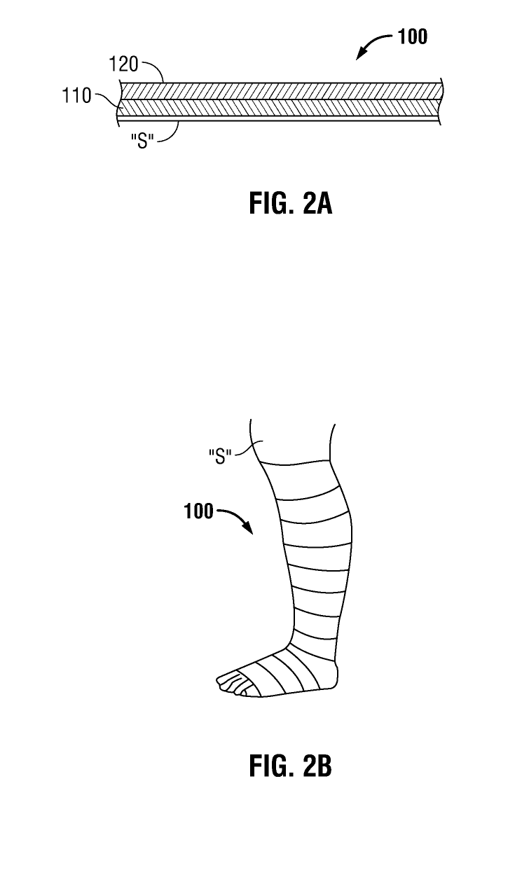

Referring now to FIGS. 2A and 2B, a compression bandage system 100 in accordance with further aspects of the invention is illustrated. Compression bandage system 100 can include a base layer 110 is positioned between skin “s” and a compression layer 120. Any of the variants discussed herein relative to the foam tape may be utilized as base layer 110. Base layer 110 may be applied to skin “s” of a limb, such as a leg (FIG. 2B). In use, for example, base layer 110 can be wrapped around the skin “s” such that adhesive layer 16 can be placed in contact with the skin “s”, as described above. In some embodiments pertinent to some aspects of the invention, the base layer 110 may be overlapped during placement thereof on skin “s”. Foam layer 12 can thus provide at least partially cushion to the skin “s” prior to the placement of an additional dressing layer, such as compression layer 120, over base layer 110. Compression layer 120 is typically an elastic dressing that provides sustained compression over at least a predetermined period.

Sterilizing any of the articles or components thereof can be performed by exposing such articles or components to a sterilizer or a sterilizing environment to at least partially inactivate any microorganisms. Sterilizing can involve any suitable technique that provides a desired level of sterility, such as a desired sterility assurance level, including, for example, any one or more of physical processes such as steaming, autoclaving, heating, chemical processes such as exposure to agents such as hydrogen peroxide, ethylene oxide, ozone, silver ions, or other oxidizing compounds such as sodium hypochlorite, irradiation processes such as exposure to gamma rays, electron beams, ultraviolet light and x-ray energy, and combinations thereof. Further embodiments pertinent to some aspects of sterilization may involve exposing the roll, tape, or components thereof to render such articles suitable under regulatory mandates. For example, the various embodiments or components thereof of the invention can be sterilized to conform with sterilization standards of medical devices as set forth by the International Organization for Standardization including, for example, any of ISO 11135 for ethylene oxide sterilization for medical devices, ISO 11137 for gamma and e-beam sterilization for medical devices, and ISO 17665 for steam sterilization for medical devices.

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments.

As used herein, the term “plurality” refers to two or more items or components. The terms “comprising,” “including,” “carrying,” “having,” “containing,” and “involving,” whether in the written description or the claims and the like, are open-ended terms, to mean “including but not limited to.” Thus, the use of such terms is meant to encompass the items listed thereafter, and equivalents thereof, as well as additional items. Only the transitional phrases “consisting of and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name, but for use of the ordinal term, to distinguish the claim elements.

What is claimed is:

1. A method of preparing a wound dressing, comprising providing a tape comprising a foam layer having a first surface and an opposite surface, an adhesive layer disposed in a predetermined adhesive pattern on at least a portion of the first surface of the foam layer, a breathable layer disposed on at least a portion of the opposite surface of the foam layer, and a release layer disposed on at least one of the breathable layer and the adhesive layer.

2. The method as in claim 1, wrapping at least a portion of the tape around a core to produce a foam roll.

3. The method as in claim 2, wherein the foam layer is comprised of polyurethane with open cells, and wherein the release layer is comprised of a material selected from the group consisting of a wax, a polyolefin, a silicone, a vinyl polymer, and combinations thereof.

4. The method as in claim 1, wherein providing the tape comprises introducing at least one therapeutic agent in the foam layer.

5. The method as in claim 1, wherein providing the tape comprises disposing the adhesive layer on the at least a portion of the first surface of the foam layer in a discontinuous adhesive pattern.

6. The method as in claim 1, wherein providing the tape comprises disposing a material selected from the group consisting of a silicone, an acrylic, and a natural rubber on the at least a portion of the first surface of the foam layer to produce the predetermined adhesive pattern.

7. The method as in claim 1, further comprising sterilizing the tape.

8. The method as in claim 1, wherein the release layer comprises a material selected from the group consisting of a wax, a polyolefin, a silicone, a vinyl polymer, and combinations thereof

9. A foam tape wrapped around a core, comprising:
an absorbent foam layer including a plurality of pores, the absorbent foam layer having a first surface and a second surface;
an adhesive layer disposed in a predetermined pattern on at least a portion of the first surface of the absorbent foam layer; and
a breathable layer disposed on at least a portion of the second surface of the absorbent foam layer.

10. The foam tape as in claim 9, further comprising a release layer disposed on at least a portion of the breathable layer.

11. The foam tape as in claim 10, wherein the absorbent foam layer comprises polyurethane.

12. The foam tape as in claim 10, wherein the release layer is comprised of a material selected from the group consisting of a wax, a polyolefin, a silicone, a vinyl polymer, and combinations thereof.

13. The foam tape as in claim 9, further comprising at least one therapeutic agent in the absorbent foam layer.

14. The foam tape as in claim 9, wherein the adhesive layer is disposed on the at least a portion of the first surface of the absorbent foam layer in a discontinuous predetermined pattern.

15. The foam tape as in claim 9, wherein the adhesive layer is comprised of a material selected from the group consisting of a silicone, an acrylic, and a natural rubber.

16. The foam tape as in claim 9 having a thickness in a range of from about 0.1 inch to about 0.3 inch.
